(19) United States
(12) Patent Application Publication
(10) Pub. No.: US 2021/0000642 A1
(43) Pub. Date: Jan. 7, 2021

(54) Thermal Body Suit

(71) Applicant: Shawn L. Stewart, Hot Springs Village, AR (US)

(72) Inventor: Shawn L. Stewart, Hot Springs Village, AR (US)

(21) Appl. No.: 17/024,324

(22) Filed: Sep. 17, 2020

Related U.S. Application Data
(63) Continuation of application No. 14/696,779, filed on Apr. 27, 2015.
(60) Provisional application No. 62/065,796, filed on Oct. 20, 2014.

(51) Int. Cl. A61F 7/00

(52) U.S. Cl. CPC A61F 7/007; A61N 5/0625; A61F 2007/0094

(57) ABSTRACT

A thermal body suit that provides heat therapy to selected areas of the body includes a garment having an outer layer, an insulation layer, a middle layer, and an inner layer, such that the insulation layer and the middle layer are disposed between the outer layer and the inner layer. The garment includes a pair of closure fasteners on opposing sides that each extend from the collar to the lower torso portion of the garment. Multiple vents are disposed on the outer layer of the garment for air circulation. The insulation layer includes a plurality of FIR coils operably connected to a power supply. A controller includes an input mechanism configured receive instructions for selecting a desired temperature of the plurality of FIR coils. In this way, the wearer can selectively apply heat to the desired areas of the body while wearing the garment.

### CROSS REFERENCE TO RELATED APPLICATIONS

This application is a continuation-in-part of U.S. patent application Ser. No. 14/696,779 filed on Apr. 27, 2015, which claims the benefit of Provisional Patent Application No. 62/065,796 filed on Oct. 20, 2014. The above identified patent applications are herein incorporated by reference in their entirety to provide continuity of disclosure.

### BACKGROUND OF THE INVENTION

The present invention generally relates to a body suit for providing heat therapy to a user. More specifically, the present invention provides to a form-fitting unitary body suit having an outer layer, multiple middle layers including an insulation layer, and an inner layer. Multiple Far Infrared Radiation (hereafter “FIR”) coils are disposed within the insulation layer. The FIR coils provide the desired level of heat to a user to help relieve pain from arthritis and fibromyalgia by helping to assist in healthy blood flow.

Many people suffer from years of chronic, nagging pain due to joint, bone, and muscle related stress, yet cannot afford surgery which may address the fundamental cause of the pain. Arthritis, fibromyalgia, and frequent stress on bones, joints, and muscles from sports-related activities, contribute to the issues people suffer on a day-to-day basis, and their quality of life diminishes. These musculoskeletal disorders are characterized by widespread pain, fatigue, sleep, memory and mood issues. Many people who have fibromyalgia also have tension headaches, temporomandibular joint (TMJ) disorders, irritable bowel syndrome, anxiety and depression. While there is no cure for fibromyalgia or arthritis, a variety of medications can help control symptoms. Exercise, relaxation and stress-reduction measures also may help.

Alternative therapies have been developed to help alleviate problems caused by musculoskeletal disorders. These include acupuncture, hypnosis, physical therapy, frequency modulation, brainwave optimization, chiropractic, vitamins and supplements, guided meditation, herbal packs and a host of other therapies. Many people are able to reduce their symptoms with a combination of exercise, medication, physical therapy, relaxation and heat therapy, especially heat therapy and Infrared heat therapy. By promoting vasodilation and expanding the blood vessels, heat therapy increases blood flow. Increased circulation brings more oxygen and nutrients to the affected areas and accelerates removal of waste products. Increased metabolic rate accelerates chemical reactions involved in tissue healing and increased oxygen-hemoglobin dissociation increases oxygen delivery to the tissues.

In addition to rushing fresh blood to the damaged tissue, heat therapy helps to flush toxins and other wastes away from the area. Therapeutic heating reduces pain by activating thermo-receptors to block the perception of pain and by promoting vasodilatation in relatively ischemic tissues. Therapeutic heating contributes to increasing range of motion by increasing soft tissue extensibility. This leads many people to using treatments such as heating pads, wraps, and warm baths to relieve pain. These remedies, however, do not provide total body relief that is continuous for extended periods of time. In view of the above concerns, it is desirable to provide a thermal body suit that utilizes FIR coils to selectively apply heat to various parts of the body.

Devices have been disclosed in the known art that relate to thermal body suits that apply heat to particular areas of the wearer's body. However, the devices have several drawbacks. For example, these devices lack a particular fastening mechanism that makes it easy to wear or take off the garment as needed. Additionally, these devices lack FIR coils disposed within a particular layer of a layered garment construction. The FIR coils of the present invention, combined with their positioning within the insulation layer, provide a more effective mechanism for applying therapeutic heat.

In light of the devices disclosed in the known art, it is submitted that the present invention substantially diverges from the prior art and consequently it is clear that there is a need in the art for an improvement to existing thermal body suits. In this regard the instant invention substantially fulfills these needs.

### SUMMARY OF THE INVENTION

In view of the foregoing disadvantages inherent in the known types of thermal body suits now present in the prior art, the present invention provides a new and improved thermal body suits wherein the same can be utilized for providing convenience for the user when treating musculoskeletal disorders such as fibromyalgia, arthritis, and osteoporosis, as well as joint and muscle pain.

In one embodiment, the thermal body suit includes a garment having an outer layer, an insulation layer, a middle layer, and an inner layer, wherein the insulation layer and the middle layer are disposed between the outer layer and the inner layer. The garment includes a first closure fastener extending from a collar of the garment to a lower torso portion of the garment on a first side of the garment, as well as a second closure fastener extending from the collar of the garment to the lower torso portion of the garment on a second side of the garment. Multiple vents are disposed on the outer layer of the garment for providing airflow. The garment further includes a plurality of FIR coils disposed within the insulation layer and operably connected to a power supply. The thermal body suit includes a controller having an input mechanism configured to receive instructions for selecting a desired temperature of the plurality of FIR coils. A USB port is disposed on the outer layer of the garment, wherein the USB port is in electrical communication with the FIR infrared coils. In this way, the USB port connection allows the user to provide power or heat instructions to the coils.

It is therefore an object of the present invention to provide a new and improved thermal body suit that has all of the advantages of the prior art and none of the disadvantages.

It is another object of the present invention to provide a thermal body suit that includes a middle layer for facilitating various properties of the garment structure, such as moisture wicking, antibacterial, or other properties.

A further object of the present invention is to provide a thermal body suit that can be easily controlled by the wearer to selectively apply heat at different temperature levels to different areas of the body.

Other objects, features, and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front perspective view of an embodiment of the thermal body suit.

FIG. 2 shows a cross-sectional view of an embodiment of the thermal body suit detailing the layered construction.

FIG. 3 shows a perspective view of an embodiment of the thermal body suit in use, with a callout image showing the FIR coils.

FIG. 4 shows a perspective view of an embodiment of the thermal body suit detailing the USB port and an example controller.

### DETAILED DESCRIPTION OF THE INVENTION

Reference is made herein to the attached drawings. Like reference numerals are used throughout the drawings to depict like or similar elements of the thermal body suit. The figures are intended for representative purposes only and should not be considered to be limiting in any respect.

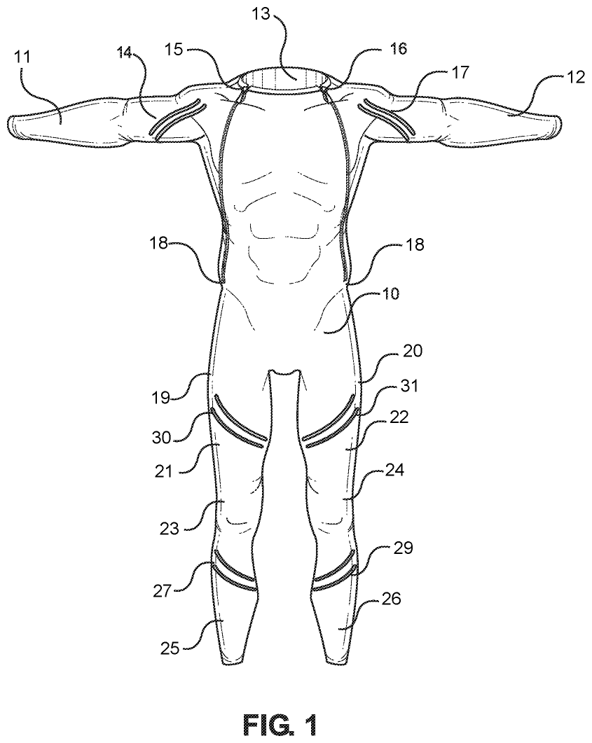

Referring now to FIG. 1, there is shown a perspective view of the thermal body suit according to one embodiment of the present invention. The thermal body suit includes a garment 10 that provides heat therapy to the wearer. The garment 10 includes a pair of arm sleeves 11, 12, a neck opening 13, and a pair of leg sleeves 25, 26 that define a unitary body suit. In the illustrated embodiment, the arm sleeves 11, 12 and the leg sleeves 25, 26 are configured to extend to the hands and legs of the wearer for maximal coverage. In other embodiments, the garment 10 can include additional hand and foot coverings. The garment 10 portion of the thermal body suit can be made in a variety of shapes and sizes to snugly fit a user. In other embodiments, the garment 10 may further include straps at the ends of the arm sleeves 11, 12 and the leg sleeves 25, 26 to provide a snug fit on the wearer.

The garment 10 includes a pair of closure fasteners 15, 16 that allow the garment 10 to be easily put on or taken off as needed. In the illustrated embodiment, the garment 10 includes a first closure fastener 15 that extends from a neck opening 13 of the garment 10 to a lower torso portion 18 of the garment 10 on a first side 19 of the garment 10, and a second closure fastener 16 extending from the neck opening 13 of the garment 10 to the lower torso portion 18 of the garment 10 on an opposing second side 20 of the garment 10. The pair of closure fasteners 15, 16 allow the front torso portion or panel of the garment 10 to be easily folded downwardly from the neck opening 13 and outwardly from the front side of the garment 10 to provide an enlarged opening that allows the user to quickly and easily put on or take off the garment 10 as needed. In the shown embodiment, the first and second closure fasteners 15, 16 include a zipper. However, in alternate embodiments, the fasteners 15, 16 can include hook and loop material, snaps, magnets, or other suitable kinds of fasteners. The dual zipper arrangement particularly allows the user to fasten or unfasten the garment 10 with a single hand and limited movement. The reduced movement requirements provided by the dual zipper configuration can be beneficial for individuals who are wearing the garment 10 to alleviate pain, as less potentially pain-inducing movement is required to put on or take off the garment 10. Further, the stopping of the fasteners 15, 16 at the lower torso portion 18 of the garment 10 allows the majority of the garment 10 to remain connected to itself for easier handling, folding, and storage.

The garment 10 further includes a plurality of vents disposed on its outer layer. The vents facilitate air flow for comfort by preventing overheating and helping to reduce the buildup of unwanted moisture that can occur when the body is heated, through sweat or condensation, as examples. In the shown embodiment, the vents make up individual vent groupings positioned at key locations on the garment 10. Each vent grouping includes a pair of vents evenly spaced and oriented parallel to each other, which facilitates maximal airflow. In the shown embodiment, the garment 10 includes a first arm vent grouping 14 disposed on the first arm sleeve 11 and a second arm vent grouping 17 disposed on the second arm sleeve 12. The arm vent groupings 14, 17 extend across the underarm and bicep area of the arms to facilitate a drying airflow to parts of the body wear moisture buildup is likely to occur. In the shown embodiment, the garment 10 also includes a first upper leg vent group 30 disposed above a knee portion 23 of the first leg sleeve 25 and a first lower leg vent grouping 27 disposed below the knee portion 23 of the first leg sleeve 25. In a symmetrical manner, the opposing second leg sleeve 20 also includes a second upper leg vent group 31 disposed above a knee portion 24 of the second leg sleeve 26 and a second lower leg vent grouping 29 disposed below the knee portion 24 of the second leg sleeve 26. This positioning allows the knee portions 23, 24 to bend while the vent groupings maintain their structure and shape for maximal effectiveness. In some embodiments, the individual vents can include their own closure fasteners which would allow the wearer to customize the airflow to their comfort.

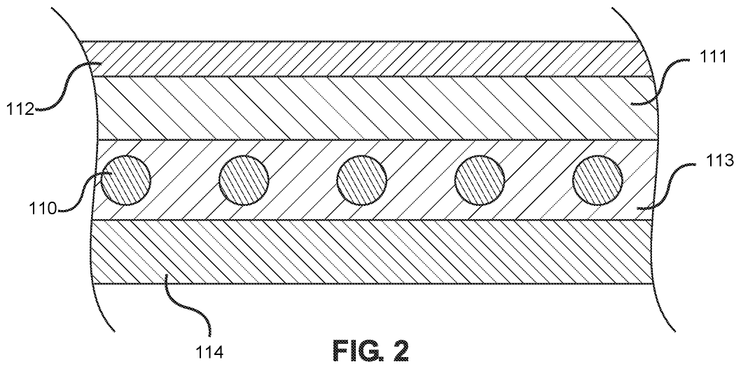

Referring now to FIG. 2, there is shown a cross-sectional view of an embodiment of the thermal body suit detailing the layered construction. The thermal body suit includes an outer layer 112, an inner layer 114, a middle layer 111, and an insulating layer 113, wherein the middle layer 111 and the insulating layer 113 are positioned between the outer layer 112 and the inner layer 114. The insulation layer 113 includes a plurality of FIR coils 110 disposed therein. Positioning of the FIR coils 110 within the insulation layer 113 allows the heat they generate to be retained and allows the overall heating system to operate more efficiently. In one embodiment, the outer layer 112 can include a neoprene material to provide thermal insulation, abrasion resistance, buoyancy. The inner layer 114 contacts the skin or any other worn garments of the user and can be made from a soft-touch material.

The middle layer 111 can include various materials depending on a desired property the material provides to the garment. For example, in one embodiment, the middle layer 111 includes a secondary layer of insulating material that helps to further retain heat for the wearer. In another embodiment, the middle layer 111 includes a bactericidal material, such as copper or silver containing materials, which can help to prevent the buildup of bacteria or other contaminants on or in the thermal body suit or its wearer. In yet another embodiment, the middle layer 111 can include a reflective material, such as a reflective metallic coating, which helps to direct heat toward the wearer and maximize efficiency of the thermal body suit. Still, in another embodiment, the middle layer can include a moisture wicking material which absorbs excess moisture. The moisture wicking material also allows the collected moisture to evaporate via the built-in vents of the garment 10.

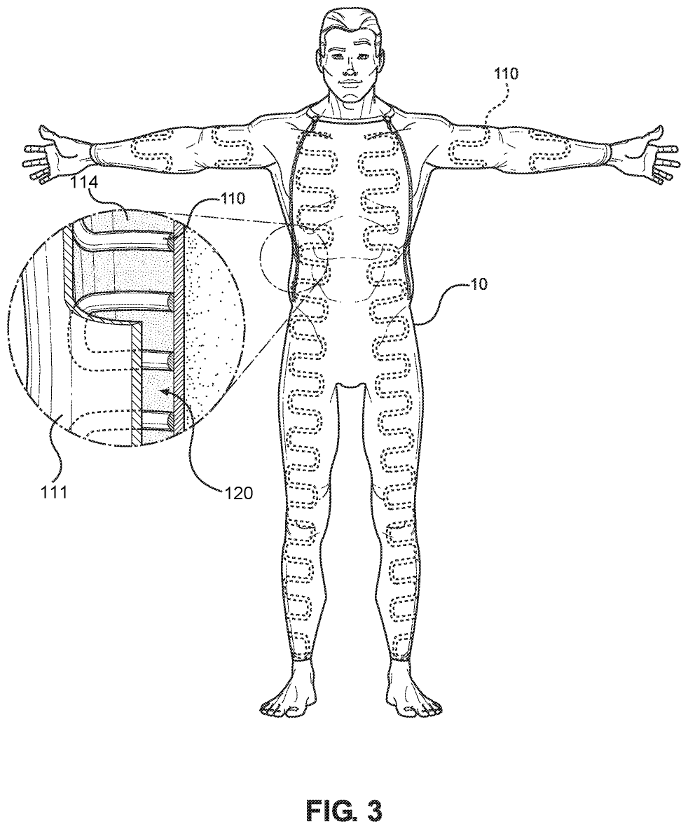

Referring now to FIG. 3, there is shown a perspective view of an embodiment of the thermal body suit in use, with a callout image showing the FIR coils. The garment 10 includes a plurality of FIR coils 110 disposed in the insulation layer. To prevent overheating, the inner layer 114 separates the wearer from the FIR coils 110 and prevents direct skin contact therewith. In one embodiment, the FIR coils define individual air pockets 120 within the insulation layer. The individual air pockets 120 include further insulating air that helps retain the heat generated by the FIR coils 110 within the garment. The FIR coils 110 are positioned throughout the garment 10 so that there are coils contacting the wearer's torso, arms, and legs. The FIR coils 110 produce ‘far infrared rays’, which are waves of electromagnetic energy that are invisible to the naked eye. Far infrared rays are capable of penetrating deep into the human body, where they elevate the body's temperature and help to activate major bodily functions. FIR can penetrate deeply into the tissues, generating the sensation of warmth as they do so. The FIR also produces a resonance in the body, thus generating this feeling of gentle warmth and repair.

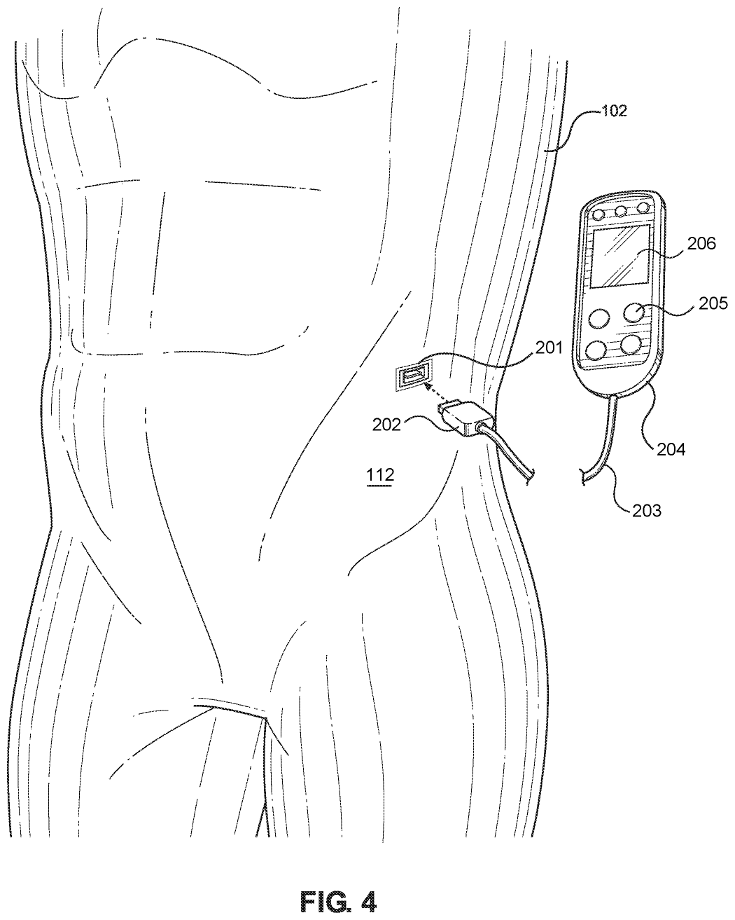

Referring now to FIG. 4, there is shown a perspective view of an embodiment of the thermal body suit detailing the USB port and an example controller. The garment 102 further includes a controller 204 adapted to control heat produced by the FIR coils. In the illustrated embodiment, the controller 204 includes an input device 205 such as a button, touchscreen, or the like, to facilitate entry of a desired temperature. The input device may further include a display panel 206 that displays temperature settings and current temperatures. The thermal body suit also includes one or more temperature sensors that communicate with the controller 204. The sensors can measure the body temperature of the wearer and the temperature of the FIR coils, and these values can be displayed on the display panel 206 of the controller 204. In some embodiments, the controller 204 also includes a timer function, such that the user can control the duration for which the FIR coils are active.

The garment 102 further includes a universal serial bus port 201 disposed on the outer layer 112 of the garment 102. The universal serial bus port 201 is in electrical communication with the FIR coils. Heat is provided through the garment 102 via the FIR coils. In some embodiments, the FIR coils are powered via an onboard rechargeable battery supply. In other embodiments, the FIR coils are directly powered via an external power source that is connected to the USB port 201, such as a connection with a wall outlet or an external battery pack. In some embodiments, the controller 204 can connected to the USB port 201 via a corresponding USB connector 202 and cable 203 for wired operation, or for powering the controller 204 and the thermal body suit with the same power supply. In other embodiments, the controller 204 can have a wireless connection to the FIR coils for wireless control.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A thermal body suit, comprising:
a garment comprising an outer layer, an insulation layer, a middle layer, and an inner layer, wherein the insulation layer and the middle layer are disposed between the outer layer and the inner layer;
a first closure fastener extending from a collar of the garment to a lower torso portion of the garment on a first side of the garment;
a second closure fastener extending from the collar of the garment to the lower torso portion of the garment on a second side of the garment;
a plurality of vents disposed on the outer layer of the garment;
a plurality of FIR coils disposed within the insulation layer and operably connected to a power supply;
a controller comprising an input mechanism configured to receive instructions for selecting a desired temperature of the plurality of FIR coils; and
a USB port disposed on the outer layer of the garment, wherein the USB port is in electrical communication with the FIR infrared coils.

2) The thermal body suit of claim, wherein first closure fastener and the second closure fasteners are zippers.

3) The thermal body suit of claim 1, further comprising a temperature sensor configured to independently measure a current temperature of a wearer of the garment and a current temperature of the plurality of FIR coils.

4) The thermal body suit of claim 1, wherein the plurality of vents comprises a plurality of individual vent groupings, wherein each vent grouping includes a pair of vents evenly spaced and oriented parallel to each other.

5) The thermal body suit of claim 4, wherein the garment comprises a first arm vent grouping disposed on a first arm sleeve of the garment and a second arm vent grouping disposed on a second arm sleeve of the garment.

6) The thermal body suit of claim 4, wherein the garment further comprises:
a first upper leg vent grouping disposed above a knee portion of a first leg sleeve of the garment and a first lower leg vent grouping disposed below a knee portion of the first leg sleeve of the garment; and
a second upper leg vent grouping disposed above a knee portion of a second leg sleeve of the garment and a second lower leg vent grouping disposed below the knee portion of the second leg sleeve of the garment.

7) The thermal body suit of claim 1, wherein the FIR coils are arranged to define a plurality of separated air pockets within the insulation layer.

8) The thermal body suit of claim 1, wherein the middle layer comprises a wicking, moisture absorbing material.

9) The thermal body suit of claim 1, wherein the middle layer defines a secondary insulation layer comprising an insulating material.

10) The thermal body suit of claim 1, wherein the middle layer comprises a heat reflecting material.

11) The thermal body suit of claim 1, wherein the middle layer comprises a bactericidal material.

12) The thermal body suit of claim 1, wherein the controller is removably secured to the USB port, wherein the controller is configured to be in electrical communication with the FIR coils when the controller is secured to the USB port via a connecting cable.
